(19) United States
(12) Patent Application Publication
(10) Pub. No.: US 2013/0030265 A1
(43) Pub. Date: Jan. 31, 2013

(54) SYSTEMS & METHOD FOR DETERMINING BLOOD COMPONENT CONCENTRATION

(71) Applicants: Justin Phillips, London (GB); Michelle Hickey, London (GB); Panayiotis Kyriacou, London (GB)

(72) Inventors: Justin Phillips, London (GB); Michelle Hickey, London (GB); Panayiotis Kyriacou, London (GB)

(73) Assignee: The City University, London (GB)

(21) Appl. No.: 13/522,159

(22) PCT Filed: Jan. 14, 2011
(86) PCT No.: PCT/EP2011/050486

§ 371 Date: Oct. 16, 2012

(30) Foreign Application Priority Data
Jan. 14, 2010 (GB) 100532.0

(51) Int. Cl. A61B 5/1455

(52) U.S. Cl. 600/310

(57) ABSTRACT

A system (1) for determining blood component concentration in vivo, the system (1) comprising: a probe (3) having an optical component and an electrical component, said optical component comprising a light source (9) for illuminating tissue (5) of a subject, said tissue (5) including a light absorbing blood component of interest, and a light detector (11) configured to detect light that has been emitted by said source (9) and has passed through said tissue (5); said electrical component comprising electrodes (13, 15) for applying an electric field across said tissue (5); and a control module (4) configured to receive signals from said light detector (11) that are representative of the intensity of light detected by the detector (11) and to receive signals from said electrodes (13, 15) that are representative of the capacitance of said tissue (5); wherein the amplitude of said signals varies periodically with the subject's cardiac cycle, and said control module (4) comprises a processor (35) operable to determine from said signals the concentration of said blood component in said tissue (5).

### FIELD

This invention relates to a system and method for determining blood component concentration. Other aspects of the present invention relate to a probe and to a control module for such a system.

The system herein disclosed is of particular utility in, and is described hereafter with particular reference to, the determination of haemoglobin concentration in blood. However, it will be appreciated by persons of ordinary skill in the art that this application is merely illustrative and that the teachings of the invention may be employed to determine the concentration of other blood components. As such, the following disclosure should not be interpreted as being limited solely to systems and methods for the determination of haemoglobin concentration in blood.

### BACKGROUND

It is known that a variety of blood disorders can be potentially serious. For example, iron-deficiency anaemia (IDA) is a relatively common disorder that affects approximately 2 billion people around the world.

In general terms, iron-deficiency anaemia occurs when a person has a sub-normal quantity of haemoglobin in their blood, typically due to a decrease in the normal size and/or number of red blood cells in their blood. The clinical definition of anaemia in adults is a blood concentration of haemoglobin of less than 12-13 mg/L of blood (usually manifested as a lack of red blood cells).

As will be appreciated, since the main function of haemoglobin is to carry oxygen from the lungs to the tissues, anaemia tends to lead to hypoxia (lack of oxygen) in organs. Mild anaemia is usually symptomless but in moderate cases, sufferers tend to exhibit symptoms of tiredness and lethargy, and in severe cases sufferers can experience dizziness, shortness of breath and cardiac arrest. Chronic anaemia can arise as a result of disease (for example, chronic diseases such as leukaemia), and acute anaemia can occur as a result of blood loss caused by trauma, during childbirth and perioperatively.

Current techniques for measuring the concentration of blood components (such as haemoglobin) are mostly invasive. In a clinical environment, such as a hospital, a physician will typically draw a sample of blood and send the sample to a laboratory where the blood is analysed by a trained technician using a hemoximeter (also otherwise known as a CO-Oximeter). Whilst such devices are typically able to provide an accurate determination of haemoglobin concentration in the sample, they are relatively expensive and need to be operated by a specially trained technician. It is also the case that as the samples need to be sent away for analysis, it is not possible to provide a real time measure of haemoglobin concentration as it typically takes a considerable amount of time for the analysis to be completed and the results returned—particularly if a haematologist reviews the results before they are returned to the physician.

As an alternative to such techniques there have previously been proposed a number of so-called “point of care” haematology analysing devices that can be operated by the physician. One such device is the HemoCue™ device manufactured and supplied by HemoCue AB, Box 1204, 262 23 Ångelholm, SWEDEN (see also: www.hemocue.com). To utilise this device, a venous blood sample (typically from a pin-prick) from an accessible site such as a finger is drawn into a disposable cuvette which is then placed in a hand-held optical analyzer that measures haemoglobin concentration spectrophotometrically.

The HemoCue™ device provides a much faster way to measure haemoglobin concentration than the aforementioned laboratory technique, but it is not without its disadvantages. For example, it has been reported (see the Technical Bulletin entitled “Total Haemoglobin Measurements: Accuracy of Laboratory Devices and Impact of Physiologic Variation” published by Masimo Corporation and available online at: http://www.masimo.co.uk/pdf/SpHb/LA65527A.pdf) that “validation literature clearly demonstrates a significant variability in capillary blood measurements compared to calibrated laboratory references. This variability is a function of both the device method and the result of using a small sample from the capillary bed where pressure can create dynamic fluid shifts. For example, if a clinician needs to push the finger to extract enough capillary blood, this forces a greater amount of plasma concentration into the blood sample and compromises the measurement.” As a consequence, results obtainable with spectrophotometric devices tend to be significantly less accurate than those obtained with a laboratory hemoximeter. Other drawback with such devices is that as they are invasive they necessarily generate biohazard waste, and can cause discomfort to the patient.

Another type of “point of care” haematology analysing device known as the I-Stat™ (available from Abbott Medical, East Windsor, N.J., USA) employs a conductometric method to determine haemoglobin concentration by calculation from a measured haematocrit. As with the HemoCue™ device, an invasive sample is required and the conductometric method of testing is prone to the same errors in measurement as spectrophotometric devices when measuring capillary blood. It has also been reported in the aforementioned Masimo Technical Bulletin that the conductivity method “has been shown to be inaccurate at haematocrits <30, or haemoglobin levels of 10 g/dL or less, limiting its ability to detect severe anaemia”.

As an alternative to the aforementioned invasive point of care devices, Masimo Corporation have developed a non-invasive device known as the Masimo Rainbow™ SET Pulse CO-Oximeter that is capable of continuously measuring total haemoglobin and other blood constituent concentrations. This device employs optical sensors and emitters and utilises more than seven wavelengths of light to acquire blood constituent data that is processed using proprietary algorithms to generate blood measurements that are displayed to the operator.

Whilst this device is an improvement over alternative invasive point of care devices, it would appear that measurements obtained are not as accurate as those obtainable with laboratory hemoximeters and would instead appear to be generally on a par with the accuracy of measurements obtained with the aforementioned HemoCue™ device -possibly because the plasma component of the blood is optically transparent and is thus difficult to detect using optical methods alone.

The present invention has been devised to address these drawbacks. In particular, aspects of the invention seek to enable real time non-invasive monitoring of blood constituents at a greater level of accuracy than existing point of care devices, in particular at a level of accuracy that is anticipated to be comparable to that provided by laboratory hemoximeters.

### SUMMARY

To this end, an aspect of the present invention provides a system for determining blood component concentration in vivo, the system comprising: a probe having an optical component and an electrical component, said optical component comprising a light source for illuminating tissue of a subject, said tissue including a light absorbing blood component of interest, and a light detector configured to detect light that has been emitted by said source and has passed through said tissue; said electrical component comprising electrodes for applying an electric field across said tissue; and a control module configured to receive signals from said light detector that are representative of the intensity of light detected by the detector and to receive signals from said electrodes that are representative of the capacitance of said tissue; wherein the amplitude of said signals varies periodically with the subject's cardiac cycle, and said control module comprises a processor operable to determine from said signals the concentration of said blood component in said tissue.

As this system employs electro-optical techniques it is pain-free and may be performed quickly and safely by an operator who has had minimal training (or indeed by a patient in the home or other non clinical environment). The proposed system avoids the risks associated with handling blood and needles, as well as the need to dispose of biohazard waste. Furthermore, once the probe has been applied to a patient, the system can be employed to provide continuous measurements - an extremely significant advantage during surgery (for example) where rapid blood loss can occur.

The varying intensity of light detected by said detector may be indicative of variations in the amount of blood component in the tissue during the cardiac cycle. The varying capacitance may be indicative of variations in the total blood volume in said tissue.

Preferably, the processor is configured to compare variations in intensity amplitude to variations in capacitance amplitude to derive a measure of the concentration of the blood component in the tissue during the cardiac cycle.

Preferably, said processor is configured to determine a ratio of ΔI/I to ΔC/C, where ΔI comprises a change in amplitude of the light intensity signal between a maximum and an adjacent minimum; I comprises the amplitude of the light intensity signal at said minimum; ΔC comprises a change in amplitude of the capacitance signal between a maximum and an adjacent minimum, and C comprises the amplitude of the capacitance signal at said minimum.

Preferably said processor is configured to determine a ratio of |Δl|/I to |ΔC|/C, where |Δl| comprises a normalised change in amplitude of the light intensity signal between a maximum and an adjacent minimum; I comprises the amplitude of the light intensity signal at said minimum; |ΔC| comprises a normalised change in amplitude of the capacitance signal between a maximum and an adjacent minimum, and C comprises the amplitude of the capacitance signal at said minimum.

In one envisaged arrangement, |ΔI| equals ΔI/I; ΔI comprises a change in amplitude of the light intensity signal between a maximum and an adjacent minimum; and I comprises the amplitude of the light intensity signal at said minimum.

In pne envisaged arrangement, |ΔC| equals ΔC/C; ΔC comprises a change in amplitude of the capacitance signal between a maximum and an adjacent minimum; and C comprises the amplitude of the capacitance signal at said minimum.

Preferably, said control module comprises means operable to drive said light source. The control module may comprise means operable to apply a varying voltage to said electrodes. The means for applying a varying voltage to said electrodes may comprise a function generator. The means for applying a varying voltage is configured to apply a sinusoidal or square wave voltage signal to said electrodes. The varying voltage signal may have a frequency of about 100 Hz.

The control module may comprise a capacitance detector coupled to said electrodes for generating a first signal representative of the capacitance of said tissue in the absence of arterial blood flow through the tissue, and a second signal representative of changes in capacitance attributable to changes in the volume of blood in the tissue. The capacitance detector may comprise a capacitance to voltage converter configured to convert capacitance signals from said electrodes into a voltage.

The control module may comprise a full wave rectifier for rectifying the voltage output by said capacitance to voltage converter. The capacitance detector may comprise a low pass filter for removing interference from voltage signals output by said full wave rectifier, said first signal The first signal may comprise the output of said filter.

The capacitance detector may comprise a high pass filter operable to isolate a high frequency component of said first signal, said high frequency component comprising said second signal. The control module may comprise an analogue to digital converter for converting analogue signals output by said capacitance detector and said light detector into digital signals for supply to said processor.

The light source may be configured to output light of a wavelength that is absorbed by the component of interest. The light source may be configured to output infrared light. The light source may be configured to output light having a peak-emission wavelength of approximately 805 nm The light source may comprise an LED.

Another aspect of the invention relates to a probe for use in the system disclosed herein, the probe comprising an optical component and an electrical component, said optical component comprising a light source for illuminating tissue of a subject and a light detector configured to detect light that has been emitted by said source and has passed through said tissue; said electrical component comprising electrodes for applying an electric field across said tissue.

The probe may comprise a first arm and a second arm, said light source and a said electrode being provided on said first arm of the probe, and said detector and the other said electrode being provided on said second arm of the probe.

The optical and electrical components may be co-located on the arms of the probe. The light source may be configured to illuminate said tissue through one electrode, and said detector may be configured to detect light through the other electrode. The electrodes may each comprise a grid.

Another aspect of the invention relates to a control module for use in the system disclosed herein, the control module being configured to receive signals from a light detector of a probe, said signals being representative of the intensity of light detected by the detector, said control module being further configured to receive signals from electrodes of said probe, said signals being representative of the capacitance of said tissue; wherein the amplitude of said signals varies periodically, and said control module comprises a processor operable to determine from said signals the concentration of a blood component in a subject's tissue.

A yet further aspect of the invention relates to a method of determining blood component concentration in vivo, the method comprising: operating a light source to illuminate tissue of a subject, said tissue including a light absorbing blood component of interest, operating a light detector to detect light that has been emitted by said source and has passed through said tissue; applying an electric field across said tissue via a set of electrodes; receiving signals from said light detector that are representative of the intensity of light detected by the detector and receiving signals from said electrodes that are representative of the capacitance of said tissue; wherein the amplitude of said signals varies periodically with the subject's cardiac cycle, and operating a processor to determine from said signals the concentration of said blood component in said tissue

Another aspect of the invention relates to a probe for use in a system for determining the concentration a blood component in vivo, the probe comprising: a first arm; a second arm; an optical emitter provided in one of said first and second arms, an optical detector provided in the other of the first and second arms; the optical detector being configured to detect light that has been emitted by said optical emitter and has traversed a subject's appendage placed between said first and second arms; a first electrode mounted in said first arm; and a second electrode mounted in said second arm, said first and second electrodes being configured for applying an electric field to the appendage of said subject.

The emitter and detector may be generally opposite one another. The first and second electrodes may be generally opposite one another. In one arrangement, the emitter and said first electrode may be co-located in one said arm, and said detector and said second electrode may be co-located in the other said arm. The probe may further comprise means for biasing said arms together. The biasing means may comprise a spring clip.

A further aspect of the invention relates to apparatus for determining the concentration of a blood component in vivo, the apparatus comprising: an interface for coupling the apparatus to a probe that is configured for attachment to a subject's appendage; means for receiving signals from said interface that are indicative of an intensity of light that has been shone through said appendage; means for receiving signals from said interface that are indicative of the capacitance of the appendage; and means operable to determine from said signals, a variation in intensity caused by absorption of said light by the blood component in said appendage, and a variation in capacitance due to changes in the volume of blood in said appendage, and to calculate from said intensity and capacitance variations a measure of the concentration of the blood component in said appendage.

A yet further aspect of the invention relates to a system for measuring a parameter of fluid pulsating through an object, the system comprising: a first module configured to apply an electric field across said object and to measure the capacitance of said object, said first module also having means for outputting a first signal corresponding to said capacitance; a second module configured to measure the opaqueness of said object to radiation of a particular wavelength, said second module also having means for outputting a second signal corresponding to said opaqueness; and a third module configured to receive said first and second signals and to calculate a parameter of said fluid therefrom.

The parameter may be the concentration of a particular component in said fluid.

The parameter may be the concentration in an amount of blood of: a) both haemoglobin and oxyhaemoglobin; b) white blood cells; c) plasma; or d) a medicament.

The first module may comprise first and second parts of a two part electromagnetic radiation measuring system.

The first and second parts of the radiation measuring system may comprise a radiation emitter and a radiation detector.

The radiation emitter may be configured to transmit radiation through an object and the detector may be configured to detect radiation emitted by the detector which has passed through the object.

The second module may comprise a capacitor.

The capacitor may comprise plates (which need not necessarily be flat and may be rounded or curved for example) at least one of which comprises a grid or a mesh.

The third module may comprise a processor for generating a signal which corresponds to ΔI, wherein ΔI is the reduction in intensity of radiation emitted by the emitter, the reduction being experienced as the radiation travels between the emitter and the detector.

The third module may comprise a processor configured to generate a signal which substantially corresponds to ΔC, wherein ΔC is the change in capacitance of said object when fluid pumps through it.

The third module may comprise a processor for calculating a ratio of ΔI and ΔC.

A method of measuring a parameter of fluid pulsating through an object, the method comprising the steps of: operating a first module to apply an electric field across said object and measuring the capacitance of said object; outputting a first signal from said first module corresponding to said capacitance; operating a second module to measure the opaqueness of said object to radiation of a particular wavelength; outputting a second signal corresponding to said opaqueness; and receiving said first and second signals by a third module and calculating a parameter of said fluid therefrom.

Other features, aspects and advantages of embodiments of the invention will be apparent from the following detailed description.

### BRIEF DESCRIPTION OF THE DRAWINGS

Various aspects of the teachings of the present invention, and arrangements embodying those teachings, will hereafter be described by way of illustrative example with reference to the accompanying drawings, in which:

FIG. 1 is a schematic sectional representation of a probe that has been applied to a patient's body part (in this instance a finger);

FIG. 2 is a schematic perspective view of part of the probe depicted in FIG. 1;

FIG. 3 is a schematic representation of an illustrative control module;

FIG. 4 is a schematic representation of a capacitance measuring component of the control module shown in FIG. 3;

FIGS. 5 is a schematic representations of intensity variations over time, and

FIG. 6 is a schematic representation of capacitance variations over time.

### DETAILED DESCRIPTION

It is known that during the systolic phase of the cardiac cycle (during which the heart ventricles contract to pump blood out of the heart) blood pressure in the arteries increases and as the artery walls are elastic, the volume of blood in the arteries increases accordingly. Conversely, during the diastolic phase of the cardiac cycle (where the heart and arteries relax), the arterial blood volume decreases.

Variations in arterial blood volume will of course cause variations in the quantity of red blood cells (RBCs) in the tissue through which the arteries pass, and as red blood cells absorb light it has previously been proposed to employ the principle of photoplethysmography to detect such variations. For example, pulse oximeters employ this knowledge to estimate the degree of blood oxygen saturation by illuminating tissue with light of different wavelengths and comparing the relative degree of absorption by haemoglobin and oxy-haemoglobin in the blood.

The teachings of the present invention also employ this knowledge to enable changes in the quantity of a given light absorbing blood component in tissue to be determined.

However, the present inventors have also appreciated that as the volume of blood in tissue varies throughout the cardiac cycle, then the electrical properties of that tissue will also vary throughout the cardiac cycle. In particular, the inventors have appreciated that as tissue can be considered to be a dielectric, then as the volume of blood varies so the capacitance of the tissue will vary, and further that this variation in capacitance will be related to the change in blood volume of the tissue in question. Since the blood volume in the tissue varies with arterial blood flow, it will now be appreciated that if the extent to which the amount of a light-absorbing component of the blood varies can be determined and if the extent to which the volume of the tissue varies, it then becomes possible to determine the concentration of that blood component in the blood.

With the above in mind, the present inventors have developed a system (described in detail below) that compares, in the course of the cardiac cycle, changes in the amount of light absorbed by a light absorbing blood component (which changes are representative of variations in the amount of that component in the tissue) to changes in the capacitance of the tissue (which changes are representative of variations in the total blood volume in that tissue), and from this comparison derives a measure of the concentration of the blood component in the tissue as blood is pulsed therethrough.

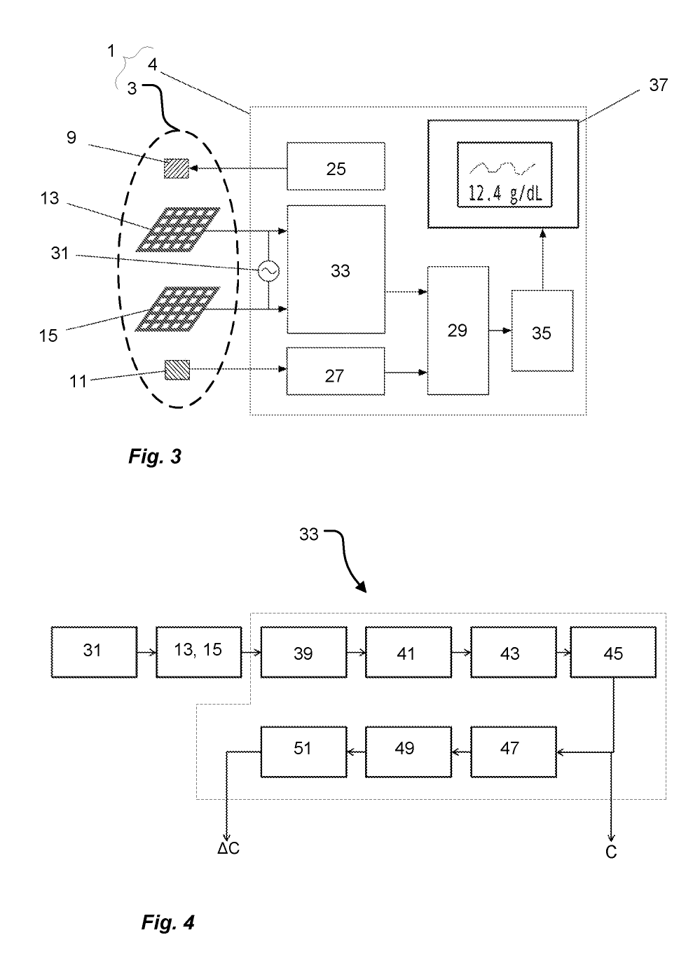

The system 1 (depicted schematically in FIG. 3 of the accompanying drawings) comprises a probe 3 and a control module 4, and as will hereafter be described is operable to non-invasively monitor blood component concentration in vivo, and on a continuous basis.

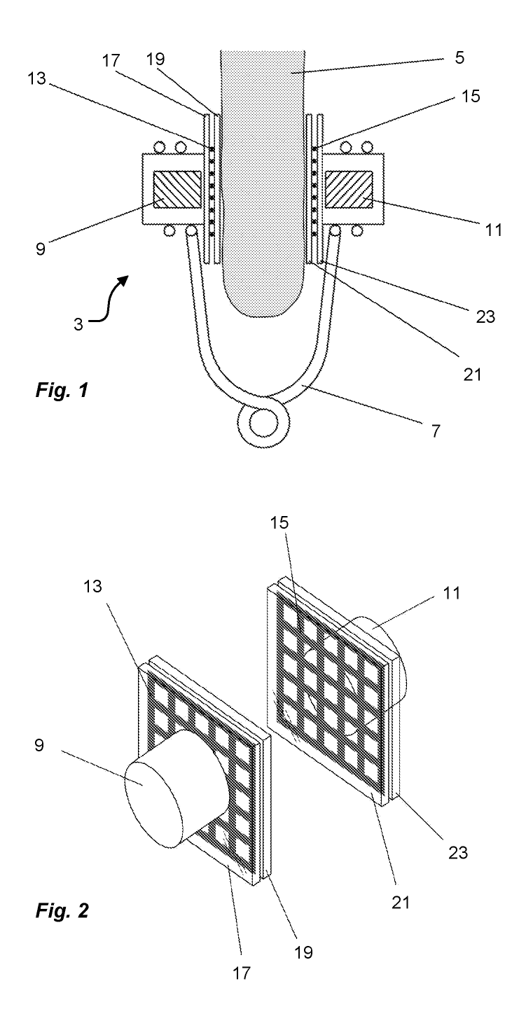

The probe (depicted schematically in FIGS. 1 and 2) is designed to fix onto an appendage of a patient's body, such as the finger 5 of the patient. Advantageously, the probe 1 could alternatively be fixed onto a patient's earlobe, thereby enabling monitoring even in circumstances where the circulatory system of the patient is operating sub-optimally (for example because the patient is in shock or is cold). In one envisaged arrangement, the probe 1 can be held in place on the appendage 5 by gentle pressure exerted by a spring-clip 7, but other equally appropriate fixing mechanisms will be apparent to persons skilled in the art.

The probe 1 comprises an optical component and an electrical component. The optical component comprises a light source 9 that is configured to emit light of an appropriate wavelength for detection of a blood component of interest. In particular the light source is configured to emit light of a wavelength that is absorbed by the component of interest. For example, in the context of haemoglobin the light source may comprise an infrared light source (such as a light emitting diode (LED)) having a peak-emission wavelength of approximately 805 nm. This wavelength is particularly advantageous in that it is an isosbestic wavelength where the absorptivity of haemoglobin is the same as the absorptivity of oxyhaemoglobin.

As shown in FIG. 1, the light source is arranged to illuminate the appendage 5, and the optical component further comprises a light detector 11 that is sensitive to the particular wavelength(s) of light emitted by the source. The light detector 11 is provided on the other side of the appendage so that it is generally opposite the source and can detect light that has travelled from the source and through the appendage. The light detector 11 may, for example, comprise a photodiode that is sensitive to light emitted by the source.

As will be appreciated by persons skilled in the art, the source illuminates the appendage of the patient with light of a wavelength that is absorbed by the blood component of interest, and the detector generates a signal, in particular a photocurrent, that varies in dependence upon the amount of incident light that is absorbed as the light traverses the appendage (and hence in dependence upon the amount of the component of interest that is in the appendage).

As will later be described in detail, the source and detector are each coupled to the control module 4 so that the control module 4 can drive the source 9 and determine the intensity of light detected by the detector 11.

The electrical component of the probe 1 comprises an anode 13 and a cathode 15 that are located, respectively, on opposite sides of the appendage 5. In a preferred arrangement, the anode and cathode are respectively co-located with the source and detector so that the optical and electrical components of the system consider the same body of tissue. It will be appreciated, however, that whilst this arrangement is preferred the electrical and optical components of the probe could merely be in close proximity to one another, for example adjacent to one another. In a preferred arrangement the anode 13 is located between the source 9 and the appendage, and the cathode 15 is located between the appendage and the detector 11. In another envisaged implementation, the anode 13 may be located between the appendage and the detector 11, and the cathode may be located between the appendage and the source 9.

In a preferred implementation where the optical and electrical components are co-located, the anode and cathode are formed as grids so as not to prevent light from flowing from the source to the detector. In another envisaged arrangement, the anode and cathode could each include an aperture in which the source and detector, respectively, are located.

In a particularly preferred arrangement, the anode grid is sandwiched between a pair of plates 17, 19 that are each transparent, at least to the particular wavelength(s) of light that are absorbed by the blood component of interest. The cathode grid is sandwiched between a similar pair of plates 21, 23. In one implementation, the plates 17, 19, 21, 23 are of glass and are capable of transmitting near-infrared radiation. The plates function both to support the anode and cathode and to protect each of them from damage.

As shown in FIG. 1, plate 17 is located between the anode 13 and the source 9, and plate 23 is located between the cathode 15 and the detector 11. Plate 19 is located between the anode 13 and the appendage 5, and plate 21 is located between the appendage 5 and the cathode 15. As will be appreciated by persons skilled in the art, the anode 13 and cathode 15 function as two plates of a variable capacitor, with the glass plates 19, 21 and the appendage 5 between the plates 19, 21 acting as the dielectric material within the capacitor. As will later be described in detail, the anode 13 and the cathode 15 are connected to the control module 4 so that the capacitance can be continuously measured.

A multi-strand cable (not shown) electrically connects the light source, the photodetector, the anode and the cathode to the control module 4. In a preferred arrangement the cable is shielded to reduce electromagnetic interference.

FIG. 3 is a schematic representation of the control module 4. It is envisaged that the control module 4 will be embodied as a relatively small and readily portable unit that can be coupled to the probe 3. For example, the control module may be configured as a hand-holdable, battery powered portable device with an integrated display.

The control module 4 contains a power supply 25 (for example a low voltage battery or suitable alternative) that powers the control system as a whole and which powers the light source 9 in the probe 3. The detector 11 outputs a signal to a circuit 27 that filters and amplifies the signal from the detector, and passes the amplified and filtered signal to an analogue to digital converter 29. A voltage source 31 draws power from the aforementioned power supply 25 and applies a varying voltage, for example a sinusoidally varying voltage, across the anode 13 and cathode 15 in the probe 3, and a dedicated capacitance detector 33 containing amplifiers and filters estimates the capacitance based on the response of the dielectric material (principally the tissue of the appendage) to the dynamic electric field between the anode 13 and cathode 15. The capacitance detector 33 outputs an analogue signal to the aforementioned analog-to-digital converter 29 that converts the analogue input signals to digital output signals. The analogue to digital converter 29 outputs to a processor 35, for example a microprocessor, a first digital signal representative of the photocurrent, and second and third digital signals representative, respectively, of changes in capacitance due to arterial blood flow and the capacitance of the appendage when no arterial flow occurs. The processor 35 calculates from these signals, in a manner described in detail below, the concentration of the blood component of interest and controls a display 37 to provide an indication of the calculated blood component concentration, preferably along with a graphical representation of trend data indicating how that component concentration has varied over time.

FIG. 4 is a more detailed representation of the aforementioned capacitance detector 33. Since the capacitance of a typical appendage is typically relatively small and the variation in capacitance due to arterial blood flow is very much smaller (for example in the order of picofarads) the capacitance detector 33 is carefully designed to reduce the potential for electromagnetic interference and to enable the very small capacitance fluctuations that are due to arterial flow to be measured.

The voltage source 31 applies a varying voltage signal as an AC carrier wave to the anode 13 and cathode 15. In an envisaged implementation the voltage source 31 comprises a function generator that is configured to generate sine or square waves (or even saw tooth waves). In a particularly preferred arrangement, the voltage source comprises a function generator configured to output a sinusoidally varying voltage signal with a frequency of about 100 kHz (this being a frequency at which the relative permittivity of various biological tissues is generally constant).

Variations in the capacitance of the appendage caused by the cardiac cycle modify the amplitude and phase of the ac carrier wave generated by the voltage source 31 to form a modified carrier wave that is passed to a capacitance to voltage converter 39 (for example of a type that is commonly used in electrical capacitance tomography ystems). The capacitance to voltage converter 39 outputs an AC voltage signal that is amplified by an amplifier 41 (typically with a gain of at least 10) and then full-wave rectified by a full wave rectifier 43. The DC output voltage of the rectifier 43 is then passed to a low pass filter 45 that is configured to filter out interference and otherwise clean up the rectified signal. In one illustrative implementation, the filter is configured as a low pass filter with a cut-off of about 22.5 Hz. The filter 45 outputs an analogue signal “C” that is indicative of the total capacitance of the appendage (i.e. the capacitance of the appendage plus a variation in capacitance due to arterial flow of blood through the appendage). However, since the variation in capacitance due to arterial flow is typically very much smaller (typically in the order of 100-1000 times smaller) than the capacitance due to the remainder of the appendage, signal “C” can be considered to be a good approximation of the capacitance of the appendage with no arterial flow.

To isolate a signal that is representative of the change in capacitance due to arterial blood low, the output from filter 45 is passed to a high pass filter 47 that passes high frequency signals, for example signals with a frequency of about 0.1 Hz or higher. The resulting signal from the high pass filter 47 is then passed to an amplifier 49 that amplifies the high frequency output signal from filter 47 (typically with a gain of at least 50) so that the amplitude of the signal can be measured. The output of the amplifier 49 may then be filtered by an optional second low pass filter 51, with a cut-off of about 22.5 Hz, that acts as an anti-aliasing filter, to remove interference and to otherwise clean up the signal. The amplifier 49 (or filter 51, if provided) outputs an analogue voltage signal “ΔC” representative of changes in capacitance due to arterial blood flow through the appendage.

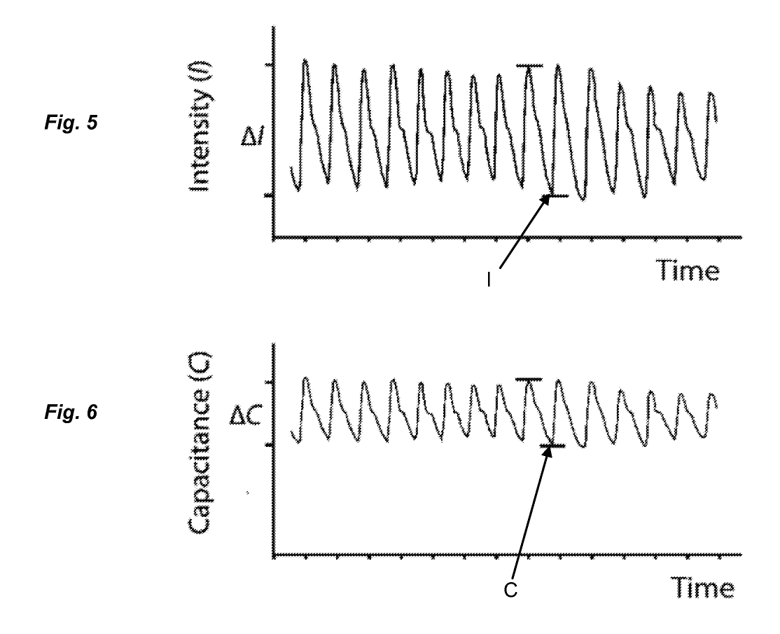

FIGS. 5 and 6 show, respectively, graphical illustrations of the variation in intensity and capacitance during the cardiac cycle.

As shown in these figures, the intensity of light detected by the detector and the capacitance vary in amplitude by a factor ΔI and ΔC, respectively, from respective minima I and C through the cardiac cycle. As aforementioned, the factor ΔI corresponds to changes in the amount of light absorbed by the light absorbing blood component in an appendage through the cardiac cycle, which changes are representative of variations in the amount of that component in the appendage. Similarly, the factor ΔC corresponds to changes in the capacitance of the appendage, which changes are representative of variations in the total blood volume in that appendage. The processor 35 is configured to compare these values and calculate a measure of the concentration of the blood component in the tissue as blood is pulsed therethrough.

As aforementioned, the processor 35 receives digital signals from the ND converter 29 that correspond to the variables C and ΔC. The processor 35 also receives a digital signal from the ND converter that varies with the light intensity detected by the detector 11, and by determining the maxima and minima of this latter signal is able to calculate the variables I and ΔI.

Considering now, by way of example, the specific example of calculating haemoglobin concentration in a patient's appendage, the processor 35 is configured to calculate a normalised (n.b. normalisation reduces inaccuracies resulting from factors such as variations in output power of the light source, and variations in sensitivity of the light detector) peak to peak intensity amplitude value |ΔI|, thus:

$$\vert Δ I \vert = \frac{Δ I}{I}$$

The processor then calculates a normalised peak-to-peak capacitance amplitude value |ΔC|, thus:

$$\vert Δ C \vert = \frac{Δ C}{C}$$

The processor then calculates a ratio of ratios R, thus:

$$R = \frac{\vert Δ I \vert / I}{\vert Δ C \vert / C}$$

and this ratio of ratios is related to the haemoglobin concentration [Hb] by the following relationship:

[Hb]=f(R)

where f is a function (that our investigations have shown to be linear or very close to linear) that is determined empirically from volunteers whose haemoglobin concentration is known, for example from a laboratory test, or from an in vitro model of perfused tissue (a so called tissue phantom) infused with blood substitute having different known haemoglobin concentrations. One or more values representative of function f may be stored in a look-up table or in memory of the processor 35.

Processor 35 is coupled to a display 37 and, in a preferred arrangement, is configured to control the display 37 to provide a continuous visual indication of the blood component concentration. The processor may also, or alternatively, be configured to control the display to provide a visual indication of variations in component concentration over time (optionally with the time being selectable by the operator) so that users of the system can readily and quickly appreciate when significant changes in concentration occur.

It will be appreciated from the foregoing that the teachings of the present invention enable the concentration of any light absorbing blood component, for example haemoglobin, to be determined, and further that the system proposed is likely to be of great utility to those involved in the practice of medicine, as well as to their patients.

It will be appreciated that whilst various aspects and embodiments of the present invention have heretofore been described, the scope of the present invention is not limited to the particular arrangements set out herein and instead extends to encompass all arrangements, and modifications and alterations thereto, which fall within the scope of the appended claims.

For example, the teachings of the present invention may—in a particularly preferred arrangement—be incorporated into a traditional known pulse oximeter by providing an additional light source that outputs red light. In such an arrangement, the oxygen saturation of the blood may be calculated by the processor 35, using conventional processing techniques, by looking at the ratio of variations in the peak to peak intensity amplitude attributable to light from the red source to variations in the peak to peak intensity amplitude attributable to light from the infrared source 9. Other useful clinical information, such as the patient's heart rate, may also be calculated by the processor 35 using conventional processing techniques (for example, from the frequency of the amplitude variation) and displayed on the display 37.

In a further modification of the system proposed that may slightly improve the accuracy of the concentration measurement, the signal output from filter 51 or amplifier 49 may be subtracted from the signal output from filter 45 to give a more accurate measure of the capacitance C of the appendage in the absence of arterial flow. In a yet further modification, it may not be necessary to normalise ΔI or ΔC, and in such an arrangement the processor would calculate R as follows:

$$R = \frac{Δ I / I}{Δ C / C}$$

It should also be noted that whilst the accompanying claims set out particular combinations of features described herein, the scope of the present invention is not limited to the particular combinations hereafter claimed, but instead extends to encompass any combination of features herein disclosed.

1-30. (canceled)

31. A system for determining blood component concentration in vivo, the system comprising:
a probe having an optical component and an electrical component, said optical component comprising a light source for illuminating tissue of a subject, said tissue including a light absorbing blood component of interest, and a light detector configured to detect light that has been emitted by said source and has passed through said tissue; said electrical component comprising electrodes for applying an electric field across said tissue; and
a control module configured to receive signals from said light detector that are representative of the intensity of light detected by the detector and to receive signals from said electrodes that are representative of the capacitance of said tissue;
wherein:
the amplitude of the signals representative of the intensity of light detected by said light detector varies periodically with the subject's cardiac cycle and is indicative of variations in the amount of blood component in the tissue during the cardiac cycle,
the amplitude of the signals from said electrodes varies with the subject's cardiac cycle and the varying capacitance is indicative of variations in the total blood volume in said tissue, and
said control module comprises a processor configured to compare variations in intensity amplitude to variations in capacitance amplitude to derive a measure of the concentration of the blood component in the tissue during the cardiac cycle.

32. A system according to claim 31, wherein said processor is configured to determine a ratio of ΔI/I to ΔC/C, where ΔI comprises a change in amplitude of the light intensity signal between a maximum and an adjacent minimum; I comprises the amplitude of the light intensity signal at said minimum; ΔC comprises a change in amplitude of the capacitance signal between a maximum and an adjacent minimum, and C comprises the amplitude of the capacitance signal at said minimum.

33. A system according to claim 31, wherein said processor is configured to determine a ratio of |ΔI|/I to |ΔC|/C, where |ΔI| comprises a normalised change in amplitude of the light intensity signal between a maximum and an adjacent minimum; I comprises the amplitude of the light intensity signal at said minimum; |ΔC| comprises a normalised change in amplitude of the capacitance signal between a maximum and an adjacent minimum, and C comprises the amplitude of the capacitance signal at said minimum.

34. A system according to claim 33, wherein |ΔI| equals ΔI/I; ΔI comprises a change in amplitude of the light intensity signal between a maximum and an adjacent minimum; and I comprises the amplitude of the light intensity signal at said minimum.

35. A system according to claim 33, wherein |ΔC| equals ΔC/C; ΔC comprises a change in amplitude of the capacitance signal between a maximum and an adjacent minimum; and C comprises the amplitude of the capacitance signal at said minimum.

36. A system according to claim 31, wherein said control module comprises means operable to drive said light source.

37. A system according to claim 31, wherein said control module comprises means operable to apply a varying voltage to said electrodes.

38. A system according to claim 37, wherein said means for applying a varying voltage to said electrodes comprises a function generator.

39. A system according to claim 37, wherein said control module comprises a capacitance detector coupled to said electrodes for generating a first signal representative of the capacitance of said tissue in the absence of arterial blood flow through the tissue, and a second signal representative of changes in capacitance attributable to changes in the volume of blood in the tissue.

40. A system according to claim 39, wherein said capacitance detector comprises a capacitance to voltage converter configured to convert capacitance signals from said electrodes into a voltage signal.

41. A system according to claim 40, wherein the control module comprises a full wave rectifier for rectifying the voltage signal output by said capacitance to voltage converter.

42. A system according to claim 41, wherein said capacitance detector comprises a low pass filter for removing interference from voltage signals output by said full wave rectifier.

43. A system according to claim 42, wherein said first signal comprises the output of said filter.

44. A system according to claim 42, wherein said capacitance detector comprises a high pass filter operable to isolate a high frequency component of said first signal, said high frequency component comprising said second signal.

45. A system according to claim 39, comprising an analogue to digital converter for converting analogue signals output by said capacitance detector and said light detector into digital signals for supply to said processor.

46. A system according to claim 31, wherein said light source is configured to output light of a wavelength that is absorbed by the component of interest.

47. A system according to claim 31, wherein said probe comprises a first arm and a second arm, and said electrodes comprise a first and a second electrode; wherein said light source and the first electrode are provided on said first arm of the probe, and said detector and the second electrode are provided on said second arm of the probe.

48. A system according to claim 47, wherein the light source and first electrode are co-located on said first arm, and the detector and second electrode are co-located on said second arm.

49. A system according to claim 31, wherein said light source is configured to illuminate said tissue through a first electrode, and said detector is configured to detect light through a second electrode.

50. A method of determining blood component concentration in vivo, the method comprising:
operating a light source to illuminate tissue of a subject, said tissue including a light absorbing blood component of interest,
operating a light detector to detect light that has been emitted by said source and has passed through said tissue;
applying an electric field across said tissue via a set of electrodes;
receiving signals from said light detector that are representative of the intensity of light detected by the detector and receiving signals from said electrodes that are representative of the capacitance of said tissue; wherein the amplitude of the signals representative of the intensity of light detected by said light detector varies periodically with the subject's cardiac cycle and is indicative of variations in the amount of blood component in the tissue during the cardiac cycle, the amplitude of the signals from said electrodes varies with the subject's cardiac cycle and the varying capacitance is indicative of variations in the total blood volume in said tissue, and
operating a processor to compare variations in intensity amplitude to variations in capacitance amplitude to derive a measure of the concentration of the blood component in the tissue during the cardiac cycle.
